(19) United States
(12) Patent Application Publication
(10) Pub. No.: US 2010/0121131 A1
(43) Pub. Date: May 13, 2010

(54) APPARATUS AND METHODS FOR STIMULATING A BODY'S NATURAL HEALING MECHANISMS

(76) Inventor: Richard A. Mathes, Byron, NY (US)

Correspondence Address: Luce, Forward, Hamilton & Scripps LLP, 2050 Main Street, Suite 600, Irvine, CA, 92614, US

(21) Appl. No.: 12/616,738

(22) Filed: Nov. 11, 2009

Related U.S. Application Data
(60) Provisional application No. 61/113,309, filed on Nov. 11, 2008.

(51) Int. Cl. A61N 2/04; A61N 5/06; A61H 1/00

(52) U.S. Cl. 600/14; 607/88; 601/47

(57) ABSTRACT

Devices, systems, and methods for inducing biologic effects in living tissue to affect the growth, repair, and maintenance behavior of the living tissue, said biologic effects induced by proximity to and exposure to light, electromagnetic, and sound energies selected according to desired responses and operated at parameters including selectable time periods, intensities, and pulse frequencies.

### CROSS REFERENCES TO RELATED APPLICATIONS

This application claims the full Paris Convention benefit of and priority to U.S. Provisional Patent Application Ser. No. 61/113,309, filed Nov. 11, 2008, the contents of which are incorporated by reference herein in its entirety, as if fully set forth herein.

### BACKGROUND

### 1. Field

This disclosure relates to therapeutic systems, methods, and products and more specifically to an apparatus and methods for stimulating natural healing mechanisms in animals

### SUMMARY

It is an object of embodiments of the present disclosure to provide methods, apparatus for inducing biologic effects in living tissue to affect the growth, repair, and maintenance behavior of the living tissue. Said biologic effects are to be induced by proximity to a control unit and exposure to monochromatic light produced by an LED array connected to said control unit, inter alia. Those skilled in the arts of cellular growth healing and maintenance shall readily grasp how the teachings of the present disclosure, as defined and described herein and claimed below, constitute an advancement in the progress of science and the useful arts, as more fully set forth below.

It is another object of embodiments of the present disclosure to provide methods for time varying emission of various electromagnetic energies including light, sound, and pulsing magnetic fields to create biologic effects in living tissue.

It is yet a further object of embodiments of the present disclosure to provide apparatus for time varying emission of electromagnetic energies to treat living tissue that is portable, inexpensive, and uncomplicated to use.

It is a further object of embodiments of the present disclosure to provide in one package a device able to provide three different electromagnetic healing modalities to be used simultaneously or individually.

It is yet another object of embodiments of the present disclosure to stimulate endogenous Nitric Oxide production, upregulate endogenous Cytochrome Oxidase activity, upregulate gene expression, upregulate protein synthesis, and stimulate osteogenesis in living bodies being treated.

It has been found that embodiments of the present disclosure create profound relaxation effects in patients being treated, speed healing, and generally decrease recovery times.

### DRAWINGS

The above-mentioned features and objects of the present disclosure will become more apparent with reference to the following description taken in conjunction with the accompanying drawings wherein like reference numerals denote like elements and in which:

FIG. 1 shows a schematic diagram of a system for stimulating a body's natural healing mechanism, according to embodiments of the present disclosure.

FIG. 2 shows a perspective view of a system including a control unit and a local unit, according to embodiments of the present disclosure.

### DETAILED DESCRIPTION

This disclosure relates to the treatment of living tissues and cells by exposing them to a mélange of electromagnetic energy modalities, each of which have been shown independently to create measurable beneficial effects in vitro and in vivo. Devices and methods according to embodiments of the present disclosure provide practitioners with aspects of simultaneous treatment with light, magnetism, and sound, which are effective for inducing effects demonstrated in the literatures, and generally serve to ameliorate and enhance recovery, at the cellular level, of organic systems from numerous challenges.

In the description that follows, embodiments will be described in reference to stimulating a body's natural healing mechanism. The present disclosure, however, is not limited to any particular application nor is it limited by the examples described herein. Therefore, the description of the embodiments that follows is for purposes of illustration and not limitation. Those skilled in the art readily understand that cellular pathways and creating down stream effect are recognized, have been approved by the U.S. FDA for human use, including, for example, the Electroceutical® brand of technology of Ivivi, Inc.® (NYSE: IVVI) (Northvale, N.J.). Numerous references are expressly incorporated herein by reference, as disclosed herein.

In both the fields of veterinary medicine and human medicine, certain problems that are recalcitrant to normal medical techniques are encountered. For instance, chronic non-healing ulcerative skin lesions and chronic osteoarthritic pain may be helped or controlled with medicine, while not being completely healed. Often, with subjects who are animals, learning disabled or otherwise challenged, clear understanding of contra-indications and best healing practices cannot be implemented. It is not necessarily non-compliance, rather not understanding the casual relationships between actions and activities and optimal healing sometimes at issue. Sometimes, what seems to be required is a gentle “nudge” to stimulate the body's natural healing mechanism. As documented, incremental boosts to healing, corrective, or maintenance cycle often are significant.

According to embodiments of the present disclosure, light, electromagnetic and sound energy, in combination, may be used to stimulate the body's natural healing mechanism. The combined light, magnetic, and sound energies may provide relaxation and stimulate tissue healing. This may be useful in veterinary applications to relax and calm an animal, while stimulating its natural healing mechanism, as heretofore uncontemplated or available commercially. Light, magnetic, and sound energies may or may not be pulsating. As can be appreciated, the light energy emitted may be absorbed and utilized at a cellular level in a mitochondrian or mitochondria set to “lubricate” and catalyze energy producing chemical reactions with a “bath” of high energy electrons.

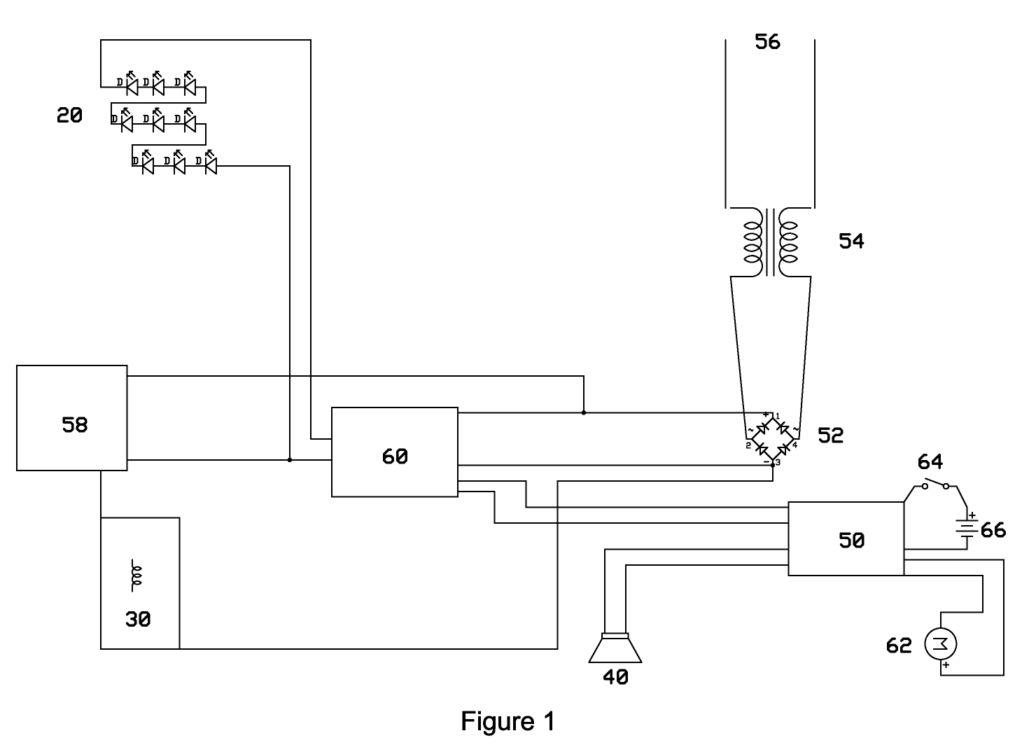

FIG. 1 shows a schematic diagram of a system and device for stimulating a body's natural healing mechanism, according to embodiments of the present disclosure. The system may include at least light source 20, electromagnet 30, and sound emitting source 40. Appropriate control and user interface components may be provided as disclosed herein.

According to embodiments, light source 20 may be configured to provide visible or infrared light used to promote tissue healing and regeneration. Using light energy for therapy provides a simple, effective, non-pharmacological medical alternative to prescription medicine or invasive surgery. Light source 20 may be one or more super-luminous light emitting diodes (LEDs), or an array thereof, to produce photons at different wavelengths in the near-infrared/visible red spectrums. Alternate methods to generate light signals, such as class III or IV lasers, may also be used. Such tools are known to those skilled in the art, omitting the need for further details of their operations.

Light emitting devices, such as light emitting diodes, may induce healing effects. Research has documented particularly effective wavelengths of monochromatic light for inducing healing effects and have elucidated protocols using LED devices for the delivery of said protocols. In particular, it has been demonstrated that LED arrays providing light at 670 nm (red) reversed the negative effect of tetrodotoxin on cytochrome oxidase in cultured neurons. Further, wound healing, laser eye injuries, and methanol induced retinal injury have been investigated, and support has been found that LED arrays delivering monochromatic light in the red to near infrared range (630-1000 nm) created significant effects namely, in vitro increases of cell growth of 140-200% in mouse derived fibroblasts, rat derived osteoblasts, and rat derived skeletal muscle cells and increase in growth of 155-171% of normal human epithelial cells. LEDs produced improvement of greater than 40% in musculoskeletal training injuries in Navy SEAL team members and decreased wound healing time in crew members aboard a US Naval submarine. LEDs also produced a 47% reduction in pain of children suffering mucositis. Also, dealing specifically with the eye, it has been demonstrated that NIR LED treatment (1) heals poisoned neurons by stimulating cytochrome oxidase activity, (2) protects against retinal damage and improves the recovery of retinal function in a rodent model of mitochondrial poison induced blindness, and (3) promotes retinal healing and improved visual function following high intensity laser induced retinal injury in adult non human primates.

It is well proven by experiment, that certain wavelengths of light have profound healing effects. Some effective wavelengths are in the visible red to near infrared range (630-1000 nm). Light in this range of wavelengths, in particular 670 nm, is known to accelerate wound healing, improve recovery from ischemic injury, and attenuate degeneration in the injured optic nerve. At the cellular level, these wavelengths create an increase in cellular proliferation and a release of growth factors from cells. At an even deeper level what seems to be driving these other processes is the activation of Mitochondrial cytochrome oxidase by the light energy. Mitochondria are the power plants of cells, and upregulating cytochrome oxidase within mitochondria provides added fuel for cells to grow, repair and maintain. While red and near infrared seem to have significant healing effects, other wavelengths should not be disregarded. For instance, UV wavelengths, while having the potential to cause damage are also necessary to produce Vitamin D in the skin. 480 nm light is important for the production of melatonin and serotonin. Embodiments of the present disclosure may provide LED arrays of different colors and wavelengths may be provided for different applications.

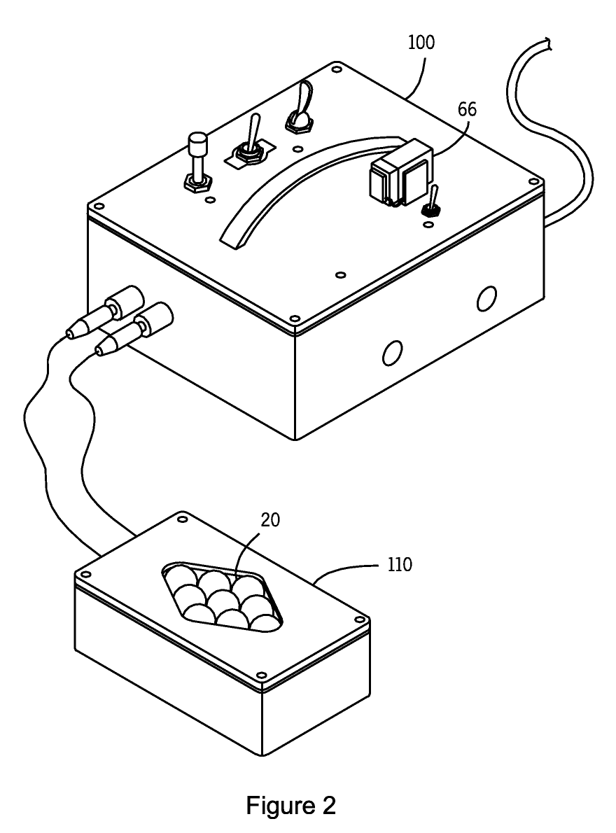

According to embodiments, LEDs of light source 20 may be arranged in clusters, as shown in FIG. 2. One or more light sources 20 may be provided in local unit 110. LED arrays may be separately provided, with each configured to provide a unique wavelength or wavelength range of light. LED colors, such as red, blue, and green may be used. One or more color/cluster may be powered at a time. Colors may be switched and alternated via quickly detachable couplers. As can be appreciated, the light source of the present disclosure (1) provides increased local circulation, (2) reduces pain, (3) improves range of motion, and (4) relaxes muscles, offering decrease of tension and spasms. Instances of each of these phenomena have been empirically demonstrated within the practice of the present applicant and the literature.

According to embodiments, electromagnet 30 may be used to generate an electromagnetic field to stimulate the production of Nitric Oxide to relax the vasculature and improve circulation. Those skilled in the art are well aware of this, and further details would be within those skill levels.

According to embodiments, applied magnetism may also facilitate healing. Systems and methods for effecting surgically noninvasive direct inductive coupling to an afflicted body region may be provided. Such systems and methods may use one or more electric voltage and concomitant current signals to create therapeutically beneficial treatment of an afflicted region. For example, enhanced repair of bone fractures, non unions, and the like have been demonstrated. Further, it has been demonstrated that magnetic fields have profound effects on inducing important endogenous biomodulating healing chemicals, particularly when the magnetic field is pulsing.

An electromagnetic field energy may be used to stimulate the production of Nitric Oxide to relax the vasculature and improve circulation. Also proven by experiment is the effect pulsing magnetic fields have on living tissue. Pulsing magnetic fields have been shown to increase bone density 20-40% in experimentally fractured bone and also increase the production of endogenous Nitric Oxide in select tissues and cells. Nitric Oxide is an important messaging molecule, vasodilator and immune system component. In vivo Nitric Oxide has a very short half life and so therefore mechanisms for enhancing it's production can be beneficial for a body trying to heal. While early experiments used strong magnetic fields on the order of 5 Tesla more, recent work has shown efficacy of picotesla fields. For example, it has been demonstrated that open wounds and sutured wounds on rats had significant improvements in healing when exposed to picotesla strength electromagnetic fields. Embodiments of the present disclosure may provide electromagnetic fields in the 100 mG range which equates to 10 microtesla (10,000,000 picotesla).

Many experiments have demonstrated that pulsing magnetic fields (“PMF”) have a beneficial effect on healing. For instance it has been shown that PMF created an increase in tensile strength of up to 69% three weeks after transaction and repair. While some of the precise mechanisms involved in PMF enhancement of healing are yet to be elucidated, there are several hypotheses working based on objective changes noted in living tissues exposed to electromagnetic fields. Among the hypothesized modes of action are induction of Heat Stress Proteins, Opioid Receptor Stimulation, and Nitric Oxide Induction.

Some researchers have even posited that electromagnetic fields may be interacting directly with electrons in DNA to stimulate biosynthesis. In addition to the aforementioned affects, previous work in the art includes U.S. Pat. No. 7,186,209 to Adey W. R., et al and U.S. Pat. No. 5,269,746 to Jerry Jacobson, et al, both of which are incorporated herein in their entirety, as if fully set forth herein. Demonstrated therein are the effects of picotesla range magnetic fields on the autonomic nervous system. For instance, fields with a strength of 0.0001-3.4 picotesla are stimulatory to the Parasympathetic aspect, and fields in the range 7.5-100 picotesla are stimulatory to the Sympathetic portion.

Researchers have determined that molecules in cells communicate by electromagnetic signaling at very low frequencies and each molecule and cell has signature frequencies at which vibrating is most comfortable. Furthermore, it has been determined that all living things function in tandem with the 24 hour cycle of the Earth's rotation and the synchronizing mechanism may not actually be within the living organism but may in fact be the geomagnetic influence of the Earth, Sun, and other planets. The Earth is known to have it's own magnetic field of about 0.5 Gauss and a signature frequency of vibration known as the Schumann resonance (fluctuating around 7-8 Hz).

As part of illness and disharmony in bodies, there may be disruptions in the normal functioning of the autonomic nervous system whether it be from electromagnetic pollution (i.e., from television, cell phones, microwaves, etc.) or chemical pollution (environmental toxins, poor diet, nutritional deficiency, recovery from trauma, etc.), and that by the method in physics known as Entrainment, homeostasis may be restored by resetting the Autonomic Nervous System. Entrainment refers to the tendency of two oscillating systems to fall into synchrony when near each other. This results from tiny exchanges of energy between the two systems that are out of phase causing one to slow down and the other to accelerate until the two are in phase.

According to embodiments, sound emitting source 40 may provide various sounds producing therapeutic effects as disclosed herein. For example, in veterinary applications, the sound emitting source may provide a sound mimicking crickets chirping.

Research has also shown that vibration, as produced by sound waves, may facilitate healing. A frequency range of 25-50 Hz creates up to 20% increase in bone strength and density, and frequencies around 100 Hz decrease symptoms of dyspnea in Chronic Obstructive Pulmonary Disease.

According to embodiments, sound emitting source 40 provides a low frequency sound signal. This low frequency sound signal may increase the local circulation of blood, lymph and the activity of the nervous system, and activate local tissue, moving old fluids out of painful areas and drawing in fresh, oxygenated fluids to enhance the healing process, reduce swelling and relax armored muscles. The low frequency sound signal reproduces the brain's natural alpha rhythm and synchronizes the signal between the brain and the nervous system, enhancing the body's natural healing process.

Sound energy may be in an audible frequency range to provide a relaxing point of focus for the conscious mind. Studies of cats and their ability to heal rapidly have looked at the influence of the sound waves produced by purring on healing. Typically cat's purrs are in a frequency range of 25-150 Hz. Researchers have found that vibrations in the 20-50 Hz range can increase bone density and speed the healing of muscles, tendons, and ligaments. Frequencies around 100 Hz can lessen the dyspnea associated with Chronic Obstructive Pulmonary Disease. Embodiments of the present disclosure may provide a low decibel sound in the about 20-25 Hz range.

Methods for non-invasively treating patients, including animals, for a variety of medical and surgical conditions including but not limited to pain, inflammation, infection, osteoarthritis, surface wounds, eye injuries, fractures, and circulatory problems (like bruising and edema) may include the application of electromagnetic energies in the form of light of various wavelengths in the visible and near infrared range, sound in the audible range, and magnetic fields ranging from 0-100 mG, pulsing at selected frequencies. As used herein, “pulse frequency” refers to the frequency at which an amplitude of a physical parameter is altered in a periodic action. A number of repetitive and oscillatory actions are known to fall within the meaning of periodic action, including sinusoidal waveforms, pulse waves, square waves, or any system operating at a given duty cycle. For example, whereas photons of light have an inherent frequency, the magnitude of emission may be managed at a given pulse frequency, such that the emission has at least one active state and at least one inactive state within a recurring period of time. According to embodiments, pulse frequency may be adjustable from 1-146 Hz and may be pre-programmed or set via controls accessible to a user.

According to embodiments, each of light source 20, electromagnet 30, and sound emitting source 40 may be provided in one or more units, such as control unit 100 or local unit 110.

For example, control unit 100 may be provided and configured to be accessible by a user and configured to be placed near a patient. Control unit 100 may be configured to house at least electromagnet 30, sound emitting source 40, controller 50, and LED driver 60. Other control and interface components may be provided on control unit 100 for operation by a user.

By further example, light may be delivered to the patient via local unit 110, configured to be applied directly to a patient. Local unit 110 may house clusters of LEDs of light source 20. The LEDs within local unit 110 may be attached via wire leads to LED driver 60 located in control unit 100. LED arrays of light source 20 that emit green, blue, red, and near infrared light may be used.

According to embodiments, circuitry of the system is constructed so that light source 20, electromagnet 30, and sound emitting source 40 may be on or off independently of each other. Switches and user interfaces may be provided to allow a user to selectively choose one or more types of therapy to be delivered. For example, switch 64 may be configured to selectively complete a circuit between controller 50 and battery 66.

According to embodiments, each parameter of the device, system, or methods may be preprogrammed, selectable from a range of options presented to a user, individually input by a user, or combinations thereof. Such parameters include, but are not limited to, pulse frequency, wavelength or intensity of light, frequency or volume of sound, magnitude of magnetic field, time of operation, which components are activated, etc.

According to embodiments, the circuitry may further provide that when a particular circuit providing one of these functions is energized, it is pulsating at the same frequency as the other circuits. Frequency may be selected by a user with an input via a multi-position switch. The multi-position switch may provide preselected frequencies determined to have resonance with particular aspects of healing. These frequencies may be selected with the multi-position switch, a single pole, single throw (“SPST”) switch, a double pole, double throw (“DPDT”) switch, another type of switch, or combinations thereof, so that one frequency or a blend of frequencies may be selected.

For example, preset pulse frequencies selectable by the multi-position switch may include:

“U”: 1.14 Hz (universal setting);
“A”: 2.28 Hz (for epithelium, skin, surface wounds, primitive undifferentiated cells);
“B”: 4.56 Hz (for visceral, gastrointestinal, metabolic, parasympathetic system);
“C”: 9.125 Hz (for motor, limb, renal, genital, sympathetic system);
“D”: 18.25 Hz (for systems showing unpaired laterality);
“E”: 36.5 (for central nervous system, spinal cord, pain and nerve conduction);
“F”: 73 (for subcortical cerebral and bone reconstruction);
“G”: 146 (for cerebral cortex);
“E” and “G”: (for analgesia);
“A” and “F”: (for regeneration); and
“C” and “G”: (for muscle relaxing).

According to embodiments, various operating frequencies for light and sound and pulse frequencies for light, sound, and magnetic fields may be provided by embodiments of the devices, systems, and methods of the present disclosure. As will be recognized by those skilled in the art, a variety of responses in a patient may be induced with given frequencies. As shown in Appendix A, a consolidated frequency list is provided from “The Electroherbalism Frequency Lists,” Third Edition (www.electroherbalism.com). Shown in Appendix A are tables of conditions and associated frequencies effective for treating the same are provided. Other frequencies are available in the art and known to those having skill. The values shown in Appendix A are frequencies measurable in hertz (Hz). According to embodiments, the frequencies of Appendix A may correspond to pulse frequencies used in devices and methods of the present disclosure.

According to embodiments, pulse frequencies provided may have an interrelationship. For example, a plurality of pulse frequencies may be provided, wherein a fundamental frequency (f) may be provided and additional frequencies are harmonics thereof (2f, 3f, 4f, etc.).

According to embodiments, controller 50 may be provided to interpret the input and initiate output of voltage and current at the selected frequency. The voltage and current is directed to chosen circuits via on/off switches. Control unit 100 containing electromagnet 30 and sound emitting source 40 is then placed next to the patient. Local unit 110 containing light source 20 is directed at any area of concern on the patient's body or any area of decreased coat density. The unit is left on to the degree of patient compliance for 15-20 minutes to provide an approximation of a dose of electromagnetic energy, as proven beneficial.

The apparatus includes, in one form of the present invention, control unit 100 that houses transformer 54, bridge rectifier 52, controller 50, and LED driver 60 interconnected with circuitry and switches. Control unit 100 may be provided with a cord to plug into a common wall socket (e.g., line voltage 56). Standard 110 volt AC household current is thereby supplied to control unit 100. Transformer 54 may be provided to step down the incoming line voltage 56 to an operable voltage (i.e., 25 volts). The alternating current may be converted to DC by bridge rectifier 52 or other conversion configuration. This current and voltage are then used to power the LEDs of light source 20 and electromagnet 30.

According to embodiments, controller 50, powered with battery 66 or other power source, may have preprogrammed frequencies that is adjusted with a multi-position switch as disclosed herein. Controller 50 may be used to control the voltage and current supplied to light source 20 and electromagnet 30 and thereby control the pulse frequency. According to embodiments, light source 20 and electromagnet 30 may be caused to have the same pulse frequency, such as where they are connected in series on in parallel with the same output of controller 50.

According to embodiments, the relative amplitude (i.e., relative gain) of light source 20 and electromagnet 30 may be managed with transistor 58. For example, a Darlington transistor may be provided and responsive to the signal provided by LED driver 60. Transistor 58 may be programmed to divide an incoming current between light source 20 and electromagnet 30 according to desired relative current to each component. For example, some currents at which electromagnet 30 may be operated may be excessive for light source 20. Thus, transistor 58 may allow a single incoming current to be properly allocated between light source 20 and electromagnet 30. Transistor 58 may allow synchronization of the pulse frequency of electromagnet 30 with the pulse frequency of light source 20 by utilizing the same incoming current.

According to embodiments, sound emitting source 40 may also be controlled by controller 50 to provide a low volume background sound, as disclosed herein.

According to embodiments, controller 50 may be any device configured to manage power and operation of other components of the system based on input provided by a user and programming stored thereon. For example, a BASIC Stamp® BS2-IC Module by Parallax Inc. (Rocklin, Calif.) or a Propeller Chip by Parallax Inc. (Rocklin, Calif.) may serve as controller 50. Controller 50 may include program interfaces for receiving and storing programmed and executable instructions and memory for storing the same.

According to embodiments, LED driver 60 may be any device configured to manage power to light source 20. LED driver 60 may regulate current over a range of load voltages, provide dimming (e.g., by means of pulse width modulation circuits), and provide control for one or more LED arrays. For example, a HV9911 DB4 by Supertex inc. (Sunnyvale, Calif.) may serve as LED driver 60.

According to embodiments, other components for management of the system in a given environment may be provided. For example, fan 62 may be provided to maintain a proper temperature of components of the system.

According to embodiments, a universal setting for a generalized treatment may also be included. This setting may provide the lowest frequency setting. As is understood by a person skilled in the art, various electromagnetic radiation sources may be used to provide the healing and relaxing attributes of the present invention. For example, electromagnetic radiation sources providing infrared, ultraviolet or ultrasonic frequencies may be used.

To provide pulsation of light, magnetic and/or sound energy, a processor/controller such as controller 50 may be used. The processor may be coupled to the light, magnetic and/or sound emitting sources and may be further configured to activate them for a predetermined period to avoid long term exposure.

According to embodiments, the light output from the high power LEDs may be about 50 lumens and the magnetic field output from the electromagnet may be about 100 milligauss. This light intensity facilitates penetration of the tissue and the total dosage of photons bathes the point of exposure. Additionally, the strength of the magnetic field ensures treatment of a larger portion of a patient's body.

According to embodiments, methods for treating a patient according to embodiments are disclosed. A method for stimulating a body's natural healing mechanism may begin by subjecting a patient to light photons from a light source for providing photodynamic therapy at a cellular level. The patient may be subjected to an electromagnetic field from an electromagnet to relax the patient's vasculature and improve blood circulation. The patient may be subjected to a sound frequency from a sound emitting source to facilitate healing and induce muscular, vascular and neural relaxation. As can be appreciated, the controller may be used to control the light source, the electromagnet and the sound emitting source for pulsating pulsate the light photons, the electromagnetic field, and the sound frequency or for emitting them simultaneously.

According to embodiments, methods for stimulating a body's natural healing mechanism may include exposing a body to light, magnetic and sound energies, in combination, to catalyze energy producing chemical reactions at a cellular level in an exposure region of a body, and induce muscular, vascular and neural relaxation. Expressly incorporated herein by reference, as if fully set forth herein, are the following U.S. Letters Patents and Publications: U.S. Pat. Nos. 4,105,017; 4,993,413; 5,033,468; 5,370,068; 5,589,863; 5,723,001; 6,149,679; 6,210,321; 6,353,763; 6,443,883; 6,604,003; 6,733,435; 6,792,315; 7,024,239; 7,008,309; 7,186,209; 2002/006554; 2005/0251279; 2006/005872; 2006/005871; 2005/0197522; 2005/0251229; 2005/0288744; 2006/0212077; 2007/0026514; 2007/0060981; 2007/0173904; 2008/0058793; 2008/0132971; 2008/0140155; and 2009/0069626.

Because of its compact size and portability, embodiments of the device and system disclosed herein may be easy to use while a patient lies on a surface, such as the floor or an examination table.

Methods of the present invention not only help in the enhancement of healing of existing conditions but also help in the prevention of new pathology via stimulatory effects on the immune system, cardiovascular system, and the growth and maintenance functions of living bodies.

According to embodiments, because of the portable nature of a device and the ease of use, it is envisioned that animal caretakers may purchase or borrow these devices to treat their animals at home and thereby take an active role in their animals healing and preventive health care.

As can be appreciated by a person skilled in the art, one or more systems of the present disclosure may be used in tandem. For example, one system or apparatus may be used on each side of a patient at a certain distance to focus the light on a particular body site to achieve particular healings.

While the apparatus and methods for stimulating a body's natural healing mechanism have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A device for treating living tissues and cells, comprising, in combination:
a controller;
a light source configured to provide pulsating light to said tissues and cells at a pulse frequency effective to stimulate Mitochondrial Cytochrome Oxidase;
an electromagnet configured to provide pulsating electromagnetic fields to said tissues and cells at the pulse frequency effective to produce endogenous Nitric Oxide; and
a sound emitting source configured to provide audible sound vibrations to said tissues and cells at a frequency effective to relax.

2. The device of claim 1, wherein the frequency of the audible sound vibrations is between about 20 and about 25 Hz.

3. The device of claim 1, wherein the pulse frequency is between 1 and 146 Hz.

4. The device of claim 1, wherein the controller is adapted to cause the light source, the electromagnet, and the sound emitting source to simultaneously emit the light, the electromagnetic field, and the audible sound vibrations.

5. The device of claim 1, further comprising an LED driver controlled by the controller and configured to deliver a pulsating electrical current to the light source and the electromagnet at the pulse frequency.

6. The device of claim 5, further comprising a transistor configured to direct a majority of the electrical current from the LED driver to the electromagnet.

7. The device of claim 1, further comprising a switch configured to cause one of a plurality of pulse frequencies to be selectable by a user.

8. A method of treating living tissues and cells, comprising, in combination, the steps of:
exposing said tissues and cells to pulsating light from a light source, whereby Mitochondrial Cytochrome Oxidase is stimulated;
exposing said tissues and cells to pulsating electromagnetic fields from an electromagnet, whereby endogenous Nitric Oxide is produced;
exposing said tissues and cells to audible sound vibrations from a sound emitting source.

9. The method of claim 8, wherein the frequency of the audible sound vibrations is between about 20 and about 25 Hz.

10. The method of claim 8, wherein a first pulse frequency of the pulsating light is between 1 and 146 Hz.

11. The method of claim 8, wherein a second pulse frequency of the pulsating electromagnetic fields is between 1 and 146 Hz.

12. The method of claim 8, wherein the pulsating light has a peak magnitude of about 40 and about 60 lumens.

13. The method of claim 8, wherein the peak magnitude of the pulsating electromagnetic fields is between about 80 and about 120 mGauss.

14. The method of claim 8, providing photodynamic therapy at a cellular level.

15. The method of claim 8, whereby the patient's vasculature is relaxed and blood circulation is improved.

16. The method of claim 8, whereby healing is facilitated by inducing muscular, vascular, and neural relaxation.

17. The method of claim 8, wherein the magnitude of the light and the electromagnetic fields pulsates at a common pulse frequency.

18. The method of claim 8, wherein the first pulse frequency and the second pulse frequency are the same.

19. The method of claim 8, further comprising controlling the light source, the electromagnet, and the sound emitting source using a controller to simultaneously emit the light, the electromagnetic field, and the audible sound vibrations.

20. A method of treating living tissues and cells, comprising, in combination, the steps of:
exposing said tissues and cells to pulsating light from a light source according to a pulse frequency;
exposing said tissues and cells to pulsating electromagnetic fields from an electromagnet according to the pulse frequency;
exposing said tissues and cells to audible sound vibrations from a sound emitting source;
whereby a mechanism of entrainment restores homeostasis by resetting the Autonomic Nervous System to achieve synchrony with the pulse frequency of the system.
